(19) United States
(12) Patent Application Publication
(10) Pub. No.: US 2018/0001007 A1
(43) Pub. Date: Jan. 4, 2018

(54) VENTRICULAR ASSIST DEVICE DRIVELINE PROTECTOR

(71) Applicant: John Stratton, Norfolk, VA (US)

(72) Inventor: John Stratton, Norfolk, VA (US)

(21) Appl. No.: 15/614,777

(22) Filed: Jun. 6, 2017

Related U.S. Application Data
(60) Provisional application No. 62/357,439, filed on Jul. 1, 2016.

(51) Int. Cl. A61M 1/12; A61M 1/10; A61M 25/02; A61M 39/02

(52) U.S. Cl. CPC A61M 1/122; A61M 1/127; A61M 39/02; A61M 1/1008; A61M 1/10; A61M 2025/0266

(57) ABSTRACT

A protective pad for encircling and protecting drivelines serving ventricular assistance devices is described. The protective pad is folded or closed around an exposed portion of a driveline just outside a body of a patient, to form a protective sleeve when installed. The protective pad includes a manual fastener such as hook and loop material, so that the protective pad is readily installed and removed. The protective pad is fabricated from a tough, slash and cut resistant polymer such as ultra-high molecular weight polyethylene, yet is flexible in ordinary use.

### RELATED APPLICATIONS

This application claims priority in accordance with 37 CF.R. 1.19(e) to U.S. Provisional Patent Application Ser. No. 62/357,439 filed for VENTRICULAR ASSIST DEVICE DRIVELINE PROTECTOR SHIELD filed Jul. 1, 2016 which is included herein in its entirety by reference.

### FIELD OF THE DISCLOSURE

The present disclosure relates to drivelines for ventricular assist devices, and more particularly, to a protective sheath for use externally to a patient having a ventricular assist device and associated driveline.

### BACKGROUND

Heart failure, often called congestive heart failure, is a condition in which the heart can no longer pump sufficient blood to the rest of the body. Heart failure is a major health problem in the U.S. with hundreds of thousands of cases diagnosed each year. There are a variety of causes for heart failure. The most common cause is coronary artery disease, which is a narrowing of the small blood vessels that supply blood and oxygen to the heart. Other causes of heart failure include congenital heart disease, heart attacks, heart valve diseases and abnormal heart rhythms (arrhythmias).

A variety of surgeries and devices have been developed to treat patients with heart failure, including coronary bypass surgery, angioplasty, heart valve surgery, addition of a pacemaker, or installation of a defibrillator. When treatments no longer work, a patient is said to be in end-stage heart failure. For patients in end-stage heart failure, a heart transplant is often the only possible treatment option. Unfortunately, there is a serious shortage of donors. The annual number of donor hearts remains around 2,000. However, the patients who are qualified to receive and need donor hearts are estimated in 2017 o be about 16,500. To compensate for this lack of donor hearts, mechanical circulation support systems have been intensively studied and developed. Such mechanical circulation support systems include artificial hearts and ventricular assist devices.

A ventricular assist device (VAD, or LVAD for left ventricular assist device) is a mechanical pump that helps a ventricle to pump blood throughout the body. The VAD pumps the blood from a weakened or diseased ventricle to the aorta or a pulmonary artery. The components of a VAD vary according to the specific device used. In general, a VAD includes a pump, connections to and from the heart, a control system and an energy supply.

The driveline or percutaneous lead is a cable for supplying power and control signals to an implantable ventricular blood pump. There is currently only one LVAD with a modular percutaneous driveline. It is the Heartmate III by Thoratec and it is presently in clinical trials. All other drivelines are continuous from the ventricular pump to the external connector that attaches to the system controller. Thoratec created the modular design because in their previous LVAD (the Heartmate II) a severely damaged driveline required complete replacement of the pump, which requires a full surgical procedure, and weeks of recovery. This is true for all other LVADs.

Cardiomyopathy is a disease that in many cases is cured only by heart transplant. Usually, this entails a wait for a donor heart. In many such cases, the need is met with a ventricular assist device. The patient may have the ventricular assist device for years or even an entire lifetime, and may be placed in a lower status of urgency on donee lists if doing well.

Driveline failure may arise from cuts, scrapes, punctures, blunt force trauma, acute bends, wire corrosion, being pinched between two objects, and other sources of damage. Also, patients having drivelines are at risk of driveline infections subsequent to driveline damage.

There exists a need to protect drivelines from damage and to protect patients from consequences of problems arising from drivelines.

### SUMMARY

The disclosed concepts address the above stated situation by providing a protective pad for encircling and protecting drivelines serving ventricular assistance devices. The protective pad is folded or closed around an exposed portion of a driveline just outside a body of a patient, to form a protective sleeve when installed. The protective pad includes a manual fastener such as hook and loop material, so that the protective pad is readily installed and removed.

The protective pad is fabricated from a tough, slash and cut resistant polymer, yet is sufficiently flexible as not to present problems arising from rigidity.

It is an object to provide improved elements and arrangements thereof by apparatus for the purposes described which is inexpensive, dependable, and fully effective in accomplishing its intended purposes.

These and other objects will become readily apparent upon further review of the following specification and drawings.

### BRIEF DESCRIPTION OF THE DRAWINGS

Various objects, features, and attendant advantages of the disclosed concepts will become more fully appreciated as the same becomes better understood when considered in conjunction with the accompanying drawings, in which like reference characters designate the same or similar parts throughout the several views, and wherein:

FIG. 1 is an environmental plan view of a protective pad in an initial stage of installation, according to at least one aspect of the disclosure;

FIGS. 2-6 show the subject matter of FIG. 1 in progressive, subsequent stages of installation;

FIG. 7 is a perspective view of the protective pad of FIG. 1;

FIG. 8 is an end view of the protective pad of FIG. 1, shown spread out and flat;

FIG. 9 is a top plan view of the protective pad of FIG. 1, shown spread out and flat; and

FIG. 10 is an end view of the protective pad as installed, and is taken along line 10-10 of FIG. 6.

### DETAILED DESCRIPTION

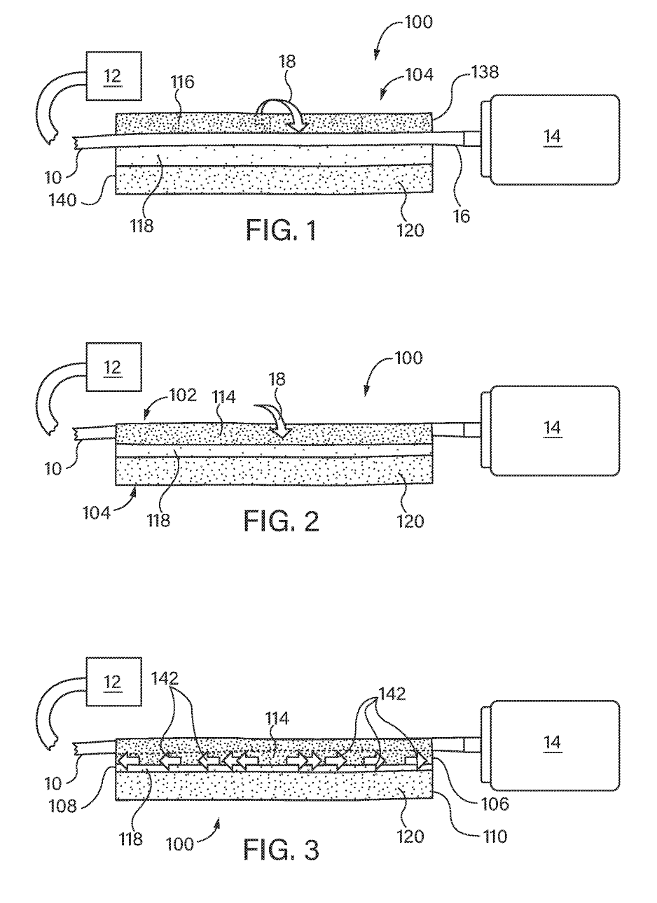

FIGS. 1-6 show a protective pad 100 being applied to a driveline 10 in progressive stages of installation. Protective pad 100 may take the form of a rectangular blanket, and is removably wrapped or folded around driveline 10. The completed installation is shown in FIG. 6, wherein after installation, protective pad 100 forms a flexible sleeve encircling a portion of driveline 10.

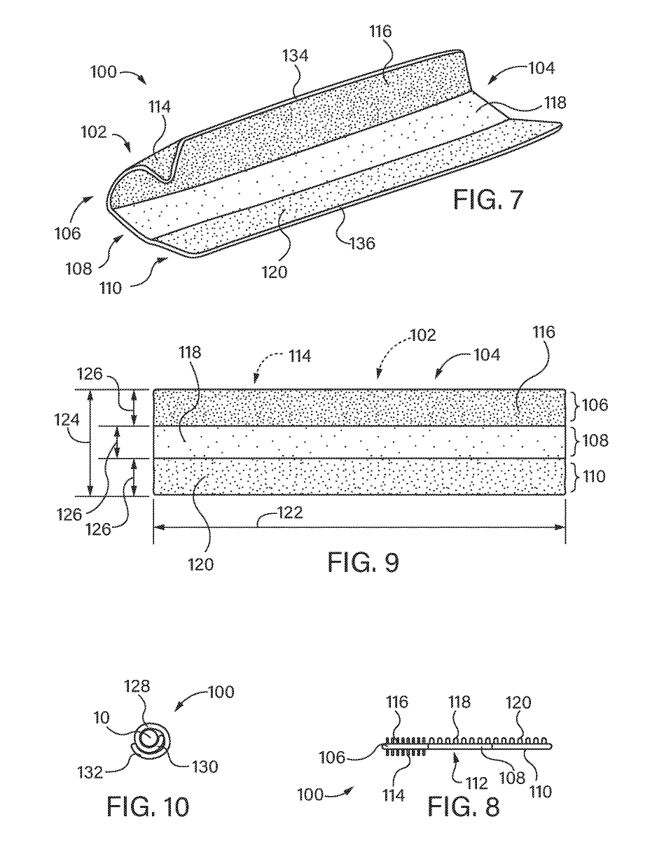

Specific construction of protective pad 100 is shown in FIGS. 7-9. Protective pad 100 for encircling and protecting drivelines (e.g., driveline 10) serving ventricular assistance devices (not shown) may include an obverse side 102 and a reverse side 104 demarcated into three parallel zones including a first zone 106, a second zone 108 abutting and parallel to first zone 106, and a third zone 110 abutting and parallel to second zone 108.

Protective pad 100 comprises a layer of slash and cut resistant material 112, a first strip of hook and loop fastener 114 of a first polarity extending along first zone 106 on obverse side 102, a second strip of hook and loop fastener 116 of the first polarity extending along first zone 106 on reverse side 104, a third strip of hook and loop fastener 118 of a second polarity extending along second zone 108 on reverse side 104, and a fourth strip of hook and loop fastener 120 of the second polarity extending along third zone 120 on reverse side 104. As will be explained hereinafter, the above described construction facilitates effective engagement of a driveline 10 on which protective pad 100 is to be installed. First, second, third, and fourth strips of hook and loop fastener 114, 116, 118, 120 need not be continuous along their respective extents, and each may be formed from more than one section of hook and loop material. The first polarity may be either hooks or loops of hook and loop material. The second polarity is the other of hooks and loops. As utilized herein, the terms “hook and loop fastener” and “hook and loop material” are used interchangeably.

As employed herein, “obverse” and “reverse” are merely for semantic distinction between the two sides or faces of protective pad 100, and should not be construed to signify that any one face will be the first to be visible.

Unless otherwise indicated, the terms “first”, “second”, etc., are used herein merely as labels, and are not intended to impose ordinal, positional, or hierarchical requirements on the times to which these terms refer. Moreover, reference to, e.g., a “second” item does not either require or preclude the existence of, e.g., a “first” or lower-numbered item, and/or, e.g., a “third” or higher-numbered item.

The layer of slash and cut resistant material may comprise ultra-high molecular weight polyethylene meeting standards ISO 13997: 1999; ASTM F-1790-05, blade cut resistance level four; EN 388:2003, with tear resistance level four and abrasion resistance level four for at least 8,000 cycles. A commercial example of such a material is Cut-Tex PRO®, a product of PPSS Group, Whitfield Business Park, Unit 1-2, Manse Lane, Knaresborough, United Kingdom, HG58BS. The slash and cut resistant material may comprise fibers other than ultra-high molecular weight polyethylene, and may be woven by high density knitting machines.

Ultra-high molecular weight polyethylene meeting the above cited standards provides requisite slash and cut protection while still being reasonably flexible.

First strip of hook and loop material 114, second strip of hook and loop fastener 116, and third strip of hook and loop material 118 may collectively cover an entirety of an area of the layer of slash and cut resistant material 112. This construction assures maximal effectiveness in engaging driveline 10 when protective pad 100 is being installed. For example, this construction will maintain snugness of contact, so that once installed, protective pad 100 will resist being displaced from its original installed position along driveline 10.

In an optional construction, first strip of hook and loop material 114, second strip of hook and loop material 116, and third strip of hook and loop material 118 may collectively cover the entirety of the area of the layer of slash and cut resistant material 112, except where a fastener (not shown) couples first strip of hook and loop material 114, second strip of hook and loop fastener 116, and third strip of hook and loop material 118 to layer of slash and cut resistant material 112. This accommodates usage of certain materials and methods of bonding hook and loop material to the layer of slash and cut resistant material 112. It would also be possible to fabricate the layer of hook and loop material to overlap or overhang its base (not shown), so as to cover an otherwise uncovered seam or zone accommodating bonding materials and methods. This construction achieves full coverage of the slash and cut resistant material, and hence optimal engagement of driveline 10, even when a seam or zone devoid of hooks and loops is necessary to accommodate bonding.

First strip of hook and loop material 114, second strip of hook and loop fastener 116, and third strip of hook and loop material 116 may be continuous from one end of protective pad 100 to an opposite end of protective pad 100. This construction optimizes grip of protective pad 100 on a driveline 10.

Referring particularly to FIG. 9, protective pad 100 may have a length 122 of about twelve inches and a width 124 of about three inches when viewed in plan in a spread-out configuration. These dimensions assure protection of driveline 10 along the most often damaged extent of the length of a driveline 10 installed in a patient.

Second strip of hook and loop material 116, third strip of hook and loop material 118, and fourth strip of hook and loop material 120 may each have a width 126 of about one inch. Adoption of a one inch width 126 assures effective sizing of hook and loop patches, yet prevents protective pad 100 from becoming objectionably large or bulky. First strip of hook and loop material 114 may have a width of about one inch, thereby also contributing to limiting protective pad 100 from becoming objectionably large or bulky.

The invention may be thought of as a protectable driveline encasement 10 for serving ventricular assistance devices (e.g., ventricular assistance device 12 in FIG. 1) and a protective sleeve. A combination of these may comprise driveline 10, and a removable sleeve dimensioned and configured to fully encircle a portion of a length of driveline 10. Protective pad 100 may serve as the removable sleeve.

Referring also to FIG. 10, in the combination of driveline 10 and the protective sleeve, the latter is longitudinally split and comprises a fastener enabling the removable sleeve to be folded around driveline 10 and over itself such that the removable sleeve when installed is three plies 128, 130, 132 thick in end view. Protective pad 100 may be thought of as a sleeve when installed on driveline 10, but slit longitudinally to result in edges 134, 136, as seen in FIG. 7. This results in a rectangular configuration which can be rolled around or folded over driveline 10, as shown in FIGS. 1-6 and 10 to reassume configuration as a sleeve. Coiling protective pad 100 around driveline 10 as shown in FIG. 10 results in very secure attachment to driveline 10.

In the combination of driveline and removable sleeve, the fastener may be a manually fastened and released fastener. This enables ready installation and removal of the removable sleeve on and from driveline 10, and obviates any need for tools. The manually fastened and released fastener may comprise hook and loop materials of complementing polarities. The hook and loop materials are readily commercially available and are in such widespread use that the public will intuitively understand their function.

The invention may also be thought of as a method of protecting driveline 10 of ventricular assistance device 12 implanted in a patient (not shown). The method may comprise placing a removable sleeve over a portion of driveline 10 exterior to the patient. Placing a removable sleeve over driveline 10 protects driveline 10 while eliminating potential undue complexity of driveline 10 which would arise from making driveline 10 self-protective. In the method, placing the removable sleeve over the portion of driveline 10 exterior to the patient may comprise placing the removable sleeve in an opened configuration in contact with a portion of driveline 10, closing the removable sleeve over the portion of driveline 10, and fastening the removable sleeve in a closed configuration encircling the portion of driveline 10. This procedure allows for an expeditiously accomplished way of securely and removably covering driveline 10.

Referring particularly to FIGS. 1-6, protective pad 100 may be installed on driveline 10 as follows. With controller and batteries 14 resting on a firm surface, driveline 10 is positioned on reverse side 104 of protective pad 100, lying along the juncture of second and third strips of hook and loop material 116, 118, as seen in FIG. 1. Preferably, end edge 138 of protective pad 100 is about half an inch from a thick portion 16 of driveline 10.

As seen in FIG. 2, protective pad 100 is rolled over driveline 10 in a direction indicated by arrow 18. Hook and loop materials of second strip 116 and of third strip 118 are pressed into contact with one another, with driveline 10 entrapped within protective pad 100.

Referring to FIG. 3, starting at the center of protective pad 100, first and second zones 106, 108 (FIG. 9) are progressively pressed into firm mutual engagement, starting at the center of protective pad 100 and progressively moving toward opposed edges 138, 140 (FIG. 1), as indicated by arrows 142.

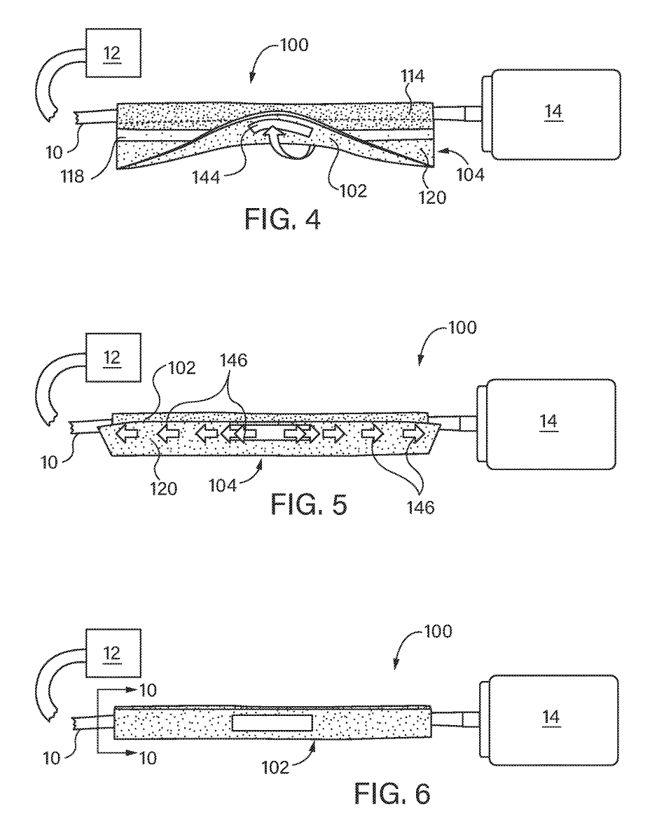

As shown in FIG. 4, a central point 144 of third zone 110 (FIG. 9) is pressed into contact with a corresponding central point of first strip of hook and loop material 114. As shown in FIG. 5, third zone 110 is pressed into full engagement with first strip of hook and loop material 114, starting at the center, and then progressively toward opposed edges 138, 140, as indicated by arrows 146.

FIG. 5 shows protective pad 100 now fully installed over driveline 10.

It should be understood that the various examples of the apparatus(es) disclosed herein may include any of the components, features, and functionalities of any of the other examples of the apparatus(es) disclosed herein in any feasible combination, and all of such possibilities are intended to be within the spirit and scope of the present disclosure. Many modifications of examples set forth herein will come to mind to one skilled in the art to which the present disclosure pertains having the benefit of the teachings presented in the foregoing descriptions and the associated drawings.

Therefore, it is to be understood that the present disclosure is not to be limited to the specific examples presented and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims.

I claim:

1. A protective pad for encircling and protecting drivelines serving ventricular assistance devices, the protective pad including an obverse side and a reverse side demarcated into three parallel zones including a first zone, a second zone abutting and parallel to the first zone, and a third zone abutting and parallel to the second zone, and comprising:
a layer of slash and cut resistant material; and
a first strip of hook and loop fastener of a first polarity, extending along the first zone on the obverse side, a second strip of hook and loop fastener of the first polarity, extending along the first zone on the reverse side, a third strip of hook and loop fastener of a second polarity extending along the second zone on the reverse side, and a fourth strip of hook and loop fastener of the second polarity extending along the third zone on the reverse side.

2. The protective pad of claim 1, wherein the layer of slash and cut resistant material comprises ultra-high molecular weight polyethylene meeting standards ISO 13997: 1999; ASTM F-1790-05, blade cut resistance level four; EN 388:2003, with tear resistance level four and abrasion resistance level four for at least 8,000 cycles.

3. The protective pad of claim 1, wherein the first strip of hook and loop material, the second strip of hook and loop fastener and the third strip of hook and loop material collectively cover an entirety of the area of the layer of slash and cut resistant material.

4. The protective pad of claim 1, wherein the first strip of hook and loop material, the second strip of hook and loop fastener and the third strip of hook and loop material collectively cover an entirety of the area of the layer of slash and cut resistant material, except where a fastener couples the first strip of hook and loop material, the second strip of hook and loop fastener and the third strip of hook and loop material to the layer of slash and cut resistant material.

5. The protective pad of claim 1, wherein the first strip of hook and loop material, the second strip of hook and loop fastener and the third strip of hook and loop material are continuous from one end of the protective pad to an opposite end of the protective pad.

6. The protective pad of claim 1, wherein the protective pad has a length of about twelve inches and a width of about three inches when viewed in plan in a spread-out configuration.

7. The protective pad of claim 6, wherein the second strip of hook and loop material, the third strip of hook and loop material, and the fourth strip of hook and loop material each has a width of about one inch.

8. The protective pad of claim 7, wherein the first strip of hook and loop material has a width of about one inch.

9. A protectable driveline for serving ventricular assistance devices and a protective sleeve, the combination comprising the driveline, and a removable sleeve dimensioned and configured to fully encircle a portion of a length of the driveline.

10. The protectable driveline of claim 9, wherein the removable sleeve is longitudinally split and comprises a fastener enabling the removable sleeve to be folded around the driveline and over itself such that the removable sleeve when installed is three plies thick in end view.

11. The protectable driveline of claim 10, wherein the fastener is a manually fastened and released fastener.

12. The protectable driveline of claim 11, wherein the fastener comprises hook and loop materials of complementing polarities.

13. A method of protecting a driveline of a ventricular assistance device implanted in a patient, the method comprising placing a removable sleeve over a portion of the driveline exterior to the patient.

14. The method of claim 13, wherein placing the removable sleeve over the portion of the driveline exterior to the patient comprises placing the removable sleeve in an opened configuration in contact with a portion of the driveline, closing the removable sleeve over the portion of the driveline, and fastening the removable sleeve in a closed configuration encircling the portion of the driveline.
